(19) United States
(12) Patent Application Publication
(10) Pub. No.: US 2023/0381111 A1
(43) Pub. Date: Nov. 30, 2023

(54) METHOD AND SYSTEM FOR ADVANCED TECHNOLOGY PACKAGING AND DELIVERY OF ACTIVE CHEMICAL INGREDIENTS

(71) Applicant: Anovotek, LLC, Barnwell, SC (US)

(72) Inventors: Daniel Alexander, Barnwell, SC (US); Don Alexander, Barnwell, SC (US)

(21) Appl. No.: 18/074,482

(22) Filed: Dec. 4, 2022

Related U.S. Application Data
(60) Provisional application No. 63/253,285, filed on Oct. 7, 2021.

(51) Int. Cl. A61K 9/50; A61K 9/08; A61K 31/00; A61K 45/06

(52) U.S. Cl. CPC A61K 9/5084; A61K 9/08; A61K 31/658; A61K 45/06

(57) ABSTRACT

A fluid system includes first microencapsulations encapsulating an active ingredient and a fluid holding the microencapsulations in suspension. A second active ingredient is in the fluid. The microencapsulations remain intact, rupture when exposed to friction, or dissolve upon passage of a period of time or based on another trigger event. A third active ingredient may be provided. A system is provided that includes a material embedded or coated with the provided fluid. The active ingredient may be a THC-free CBD isolate. An exemplary method provides exposure to a first active ingredient and a second active ingredient. Exposure to the first active ingredient and/or the second active ingredient may alleviate symptoms of arthritis, or repel insects.

### CROSS-REFERENCE TO RELATED APPLICATIONS

The present application claims priority to U.S. Provisional Patent Application No. 63/253,285, filed Oct. 7, 2021, entitled “ADVANCED TECHNOLOGY PACKAGING AND DELIVERY OF ACTIVE CHEMICAL INGREDIENTS”, which is incorporated by reference in its entirety.

### BACKGROUND OF THE INVENTION

### 1. Field of the Invention

The invention relates to the use and delivery of active ingredients to prevent premature activation, and in particular to methods and systems for application of active ingredients without premature activation.

### 2. Description of the Related Art

The topical application of aromatic chemicals has several uses. For an individual chemical or compound to impart an effect, it must be sufficiently volatile to effect transmission via the air by undergoing a phase transition from a solid or a liquid into a gas after it is applied to a surface. In common parlance, these chemicals are said to have smells or tastes. These are small molecules with a molecular weight that is typically less than 310 kg/mol, which facilitates their gaseous or semi-gaseous nature at room temperature. Many esters, linear terpenes, cyclic terpenes, and amines fall into this category. Once applied, these chemicals rapidly vaporize until there is little or none of the applied chemical remaining on the surface in its original state.

U.S. Pat. No. 10,052,277 discusses compositions that provide release of fragrance over an extended period of time. The compositions comprise a hydrophilically-modified cross-linked silicone elastomer and an acrylic rheology modifier.

U.S. Pat. No. 7,294,612 discusses a polymeric encapsulated fragrance which is suitable for use in personal care and cleaning products. In an embodiment the fragrance is encapsulated by a first polymer material to form a fragrance encapsulated polymer. The polymer encapsulated shell is then coated with a cationic polymer, preferably a cationic starch and guar.

U.S. Pat. No. 6,113,935 discusses a suspension of microcapsules in an organic liquid. The microcapsules contain an aqueous phase. The suspension is produced by interfacial polymerization in the presence of a proton transfer catalyst of a water-in-oil emulsion in which the aqueous phase contains a urea/formaldehyde or melamine/formaldehyde prepolymer.

U.S. Pat. No. 5,225,278 discusses a process for encapsulating a wide variety of target materials, including both hydrophilic and hydrophobic materials, employs condensation of two reactive compounds to form shells around core phase particles including target material dispersed in a continuous phase. One of the reactive compounds has at least two active methylene functional groups per molecule, the other being an active methylene-reactive crosslinking agent. Either type of the reactive compounds can be dispersed in the continuous phase, the other being dispersible in the core phase. Applications include controlled release microencapsulation of agriculture chemicals and biocides.

U.S. Pat. No. 8,753,676 discusses a method for the preparation of microencapsulated essential oils. The microcapsules contain essential oils or a formulation thereof and may be used for various non-agricultural applications.

U.S. Pat. No. 6,617,364 discusses thermo-expandable microspheres and the expanded microballoons, microcellular foam, or foamed composite material that results upon heating the microspheres. The thermo-expandable microsphere has a polymeric wall surrounding one or more pockets or particles of blowing agent or propellant within the microsphere. The polymeric wall may have reactive functional groups on its surface to give a fusible microsphere. When the microspheres are heated, they expand to form microballoons comprising polymeric shells surrounding one or more internal gaseous voids, and when the microspheres are expanded while in contact with each other, a microcellular foam may be formed. The foam consists of a plurality of microballoons fused together, optionally aided by functional groups present on the surface of the heated microspheres that act to crosslink the material. When microspheres are mixed with a matrix, which can optionally react with functional groups on the microsphere surface, and the resulting combination is heated, the microspheres expand to give a foamed composite material in which the microballoons may be fused or chemically crosslinked to the matrix.

U.S. Pat. No. 10,415,000 discusses a method of forming microcapsules having improved physical properties and release control, as well as the microcapsules formed by the process. The capsule wall is formed by the concurrent polymerization of monomers, oligomers, and/or prepolymers on the inside of the capsule wall, and different monomers, oligomers, and/or prepolymers on the exterior of the capsule wall.

### SUMMARY OF THE INVENTION

The present technology provides a novel and useful way of extending the effective timeframe of topically applied chemicals, such as aromatics or oils. The present technology reduces interactions between multiple chemical components until the components are activated or otherwise used, thus preserving longevity of active ingredients and allowing delivery of various chemicals with varying properties in a single product.

The present technology delivers organic and inorganic chemicals, including aromatics, flavors, essential oils and blends of various chemicals, to cause results such as cosmetic benefits, pain relief, skin improvement, dermatology treatments, wellness, aromatherapy, insect repellency. The present technology extends the effective longevity of the products and minimizes chemical interactions prior to activation or use.

Consumers often apply a topical chemical at the beginning of an activity but are then forced to reapply the same topical chemical one or more times as efficacy wains due to the relatively short period of time needed for degradation, reactions, evaporation, or vaporization to occur. The present technology extends that time by combining a series of microscopic physical and chemical processes to slowly release active ingredients in order to prolong usefulness of a single application while minimizing chemical reactions of components. The technology allows complex blends of chemicals to be co-applied while minimizing the interaction and reactivity among the various components until the chemicals are released or activated.

Previous applications have been lacking in a number of ways. Relying on chemical reactions to gradually generate active ingredients is unstable as it could result in unexpected results due to changes in ambient moisture, temperature, or other chemicals present. Other methods of suspending or encapsulating chemical reactants are somewhat effective but have a limited lifespan as a result of their singular nature. For instance, if a chemical is likely to evaporate in an hour's time when topically applied by itself, a conventional method, such as coating the chemicals in saccharides to slow down evaporation, would only extend the life of the product to two hours and still require reapplication during most activities or outings. However, the present invention combines or stacks a series of methodologies to vastly extend efficacy past the timeframe which has been available by using a single mechanical or chemical means of releasing active ingredients.

An exemplary method uses microencapsulation, though other methods may be utilized and/or combined with microencapsulation. Studies support the efficacy of insect repellents related to the efficacy of the various blends conducted both in laboratory and field experiments on three different species of mosquito, Aedes, Culex and Anopheles. Keeping the repellents separated improves efficacy and reduces the potential for mosquitoes to develop resistance.

An exemplary embodiment may be used as a bug repellent, and provides a plant-based insect repellent applied directly to outdoor furniture. When furniture is used, the present technology activates and releases a long-lasting repellent with a pleasant scent, which is safe for the family, pets, and the environment. The present technology uses essential oils as an alternative to DEET-based insect repellents, which are sticky and unpleasant-smelling synthetic chemicals applied to the skin.

The present technology provides a delivery system to create various blends of repellents (i.e. Cedarwood. Citronella and Lemongrass). The interaction between essential oils is reduced by having one or more individual essential oil encapsulated in a micro-container. Each formulation may contain two or more, for instance five or six, different essential oils. Keeping the essential oils separated in micro-containers until activated ensures that the efficacy of each repellent is not changed by chemical interaction with the other essential oils and other ingredients in the formulation. Essential oils volatilize when applied directly to a surface, but using the present technology they are held in micro-containers or complex matrices that significantly reduce the evaporation rate.

Cannabidiol (CBD) may be used alone or with other essential oils for the treatment of pain, skin problems, or other medical issues. CBD is a known and growing option for pain relief. The present technology provides blends of CBD and other active ingredients, reducing the interaction of the CBD and other active ingredients until they are released by friction. For example, CBD may be combined with menthol and copaiba for the treatment of muscle and/or joint pain. CBD may also be combined with squalene oil, and may be THC Free CBD Isolate.

The present technology provides a spray of one or more of CBD, menthol, copaiba, and squalene for pain relief and/or improved mobility. Immediate relief may be provide and/or encapsulated, delayed-release treatment. The spray may be used during the manufacture of sleeves, socks or other clothing that makes close contact with the body, and/or may be used by a consumer to recharge a previously infused piece of clothing, or to modify any clothing item and/or close fitting athletic wear.

An exemplary embodiment of the present technology provides a fluid system that includes a first plurality of microencapsulations encapsulating a first active ingredient and a fluid holding the first plurality of microencapsulations in suspension. A second active ingredient is in the fluid.

In exemplary embodiments, the first plurality of microencapsulations are configured to rupture when exposed to friction or dissolve upon passage of a period of time. In still further alternative exemplary embodiments, the microencapsulations dissolve when exposed to a different pH level, for instance an acidic environment, or when exposed to air.

In further exemplary embodiments, the second active ingredient is dissolved in the fluid. Alternatively, the second active ingredient may be encapsulated in a second plurality of microencapsulations.

The second plurality of microencapsulations may be configured to rupture when exposed to friction or dissolve upon passage of a further period of time, or dissolve in response to any other possible trigger.

In further exemplary embodiments, a third active ingredient may be dissolved in the fluid or encapsulated in a third plurality of microencapsulations. The third plurality of microencapsulations may be configured to rupture when exposed to friction, dissolve upon passage of another period of time, or dissolve in response to another trigger.

A system is provided that includes a material, which may be a textile, a woven fabric, a flexible material, or any other material. A first plurality of microencapsulations encapsulating a first active ingredient is embedded in the material or applied to a surface of the material. A second active ingredient is also provided in or on the material.

In the exemplary system, the first plurality of microencapsulations may rupture when the material is manipulated or dissolve upon passage of a period of time, or based on any other trigger.

The second active ingredient may be absorbed into the material or applied to the surface of the material. The second active ingredient may be encapsulated in a second plurality of microencapsulations, which may rupture when the material is manipulated or dissolve upon passage of a further period of time, or based on an alternative trigger.

In further exemplary embodiments, a third active ingredient is provided and is absorbed into the material or encapsulated in a third plurality of microencapsulations. The third plurality of microencapsulations may rupture when the material is manipulated or dissolve upon passage of another period of time, or based on another trigger.

The material may be used to make a knee brace, a wrist brace, an elbow brace, an ankle brace, a glove, and a sock. The first active ingredient or the second active ingredient may be a THC-free CBD isolate.

An exemplary method provides exposure to a first active ingredient and a second active ingredient. The exemplary method includes encapsulating, in a first plurality of microencapsulations, a first active ingredient. The exemplary method also includes providing a second active ingredient either 1) dissolved in a fluid holding the first plurality of microencapsulations in suspension, 2) encapsulated in a second plurality of microencapsulations, or 3) absorbed into a material in which the first plurality of microencapsulations are embedded.

The first plurality of microencapsulations may be configured to rupture when applied to a surface or dissolve upon passage of a period of time, or based on another trigger.

Exposure to the first active ingredient and/or the second active ingredient may alleviate symptoms of arthritis, or repel insects.

### BRIEF DESCRIPTION OF THE DRAWINGS

The invention is described in more detail with reference to the accompanying drawings, in which only preferred embodiments are shown by way of example. In the drawings:

FIG. 1 is a diagram illustrating chemical encapsulations without a suspension fluid and with a suspension fluid;

FIG. 2 is a diagram illustrating chemical encapsulations embedded into a mesh or fabric; and

FIG. 3 is a flowchart illustrating a method according to an exemplary embodiment of the present invention.

### DETAILED DESCRIPTION

The exemplary method provided herein enables a method to isolate the active ingredients to avoid interaction with each other including during application until they are activated.

The present technology uses a combination of methodologies in a unique way to prevent interference or premature reaction, interaction or vaporization between numerous active ingredients. These delivery methods include microencapsulation, hollow glass microspheres, porous wall hollow glass microspheres, polymer microspheres, polymer carriers, zeolites, clathrates, porous ceramics, porous silica, and mesoporous materials such as mesoporous silica. First, subordinate methods of physical barriers are used to extend the time chemicals are released into the air on a base level. Additional layers of complexity are possible with various combinations of one or more of the described delivery methods. In an exemplary embodiment, a mixture of aromatics in separate microencapsulations within a polymeric shell are provided. As each shell breaks or deteriorates, a new set of time released active topical chemicals are exposed to the air on the applied surface, dramatically extending the efficacy of a single application of the resultant product.

The invention uses the combination of microencapsulation and sub-encapsulation separation to prevent chemicals from not only evaporating, but also from interfering with one another. Chemicals like essential oils or other active components may react with the air or one another, so separating multiple ingredients prior to their release also prevents interactivity between multiple ingredients. Blending can be caused by the user, for instance by rubbing an applied surface to physically break down means of separation, or alternatively, by the natural degradation of the separation means when exposed to the elements. In either case, breaking down an external encapsulation may result in the release of interstitial chemicals as well as further encapsulated compounds (sub-encapsulations). For some of these compounds, the internal capsules may act as a shell from which the internal ingredients continue to provide beneficial properties such as insect repelling or sun protection because of their gradual release or topical availability.

One valuable functionality of the present technology is the ability to generate or manufacture blends of ingredients without expensive and time-consuming redundant testing. Because internal compounds are isolated from one another, their efficacy and precautionary traits are retained regardless of the presence of other compounds that serve other purposes. While blending multiple active compounds in a traditional manner risks chemical reactions between new combinations of ingredients, the present invention allows for each component of a broader mixture to be inaccessible to other components as a result of their respective protective shells or carriers. This minimizes chemical interactions and potential reactions between various ingredients, allowing otherwise reactive compounds to be used in concert with one another in a way that was not previously possible.

The present invention has several potential applications. For instance, the efficacy of certain insect repellants can be enhanced by creating unique combinations of both natural and synthetic repellants that are gradually released over the course of a day or longer. These blends can be easily and efficiently customized to target specific types of biting arthropods and insects that exist in different environments. Numerous varied compounds may be included to target differing pests when utilized in environments that may otherwise require numerous incompatible combinations of ingredients or products. Other applications include but are not limited to dramatically extending the life and efficacy of sanitizing and disinfecting compounds, and providing otherwise reactionary or quickly vaporizing aromatherapy compounds or perfumes over longer periods of time. Additionally, the present technology may enable topical delivery of compounds such as antiperspirants or sun blocks, and delivery over extended periods of essential oils and blends of various chemicals having desirable attributes, such as cosmetic ingredients, pain relief, skin improvement, dermatology treatments, and wellness.

Exemplary delivery methods include microencapsulation, cyclodextrins, hollow glass microspheres, porous wall hollow glass microspheres. Alternative exemplary delivery mechanisms include microencapsulation into a polymeric shell. The present technology prevents chemical blending and interference between essential oils and other components, and/or interaction with air or other external materials.

In exemplary embodiments, usage by a user operates to activate the active ingredients, by for instance, rupturing the microencapsulations by application to the skin or a surface. In further exemplary embodiments, the active ingredients may remain inside the carrier (shell) and provide beneficial properties (such as insect repellency).

The present technology provides the significant benefit of enabling different blends of components to be made and marketed without having to go through reassessment of safety, efficacy, etc., because each component is kept inside a protective shell (or carrier) minimizing chemical interactions and potential reactions.

An exemplary embodiment include an insect repellent with a custom blend of natural insect repellent products. The efficacy of insect repellents can be enhanced by creating unique blends or various natural and/or synthetic repellents. Customized blends can be made quickly and efficiently to target specific types of biting arthropods and other insects.

A further exemplary embodiment includes sanitizing and disinfecting, which may be enhanced by combining ingredients that do not interact until activated or released through use or environmental conditions.

Another exemplary embodiment is aromatherapy, in which various aromatherapy blends can be created by avoiding combining the essential oils prior to encapsulation.

The present technology creates complex mixtures of different active ingredients with the purpose of reducing interaction and preserving and enhancing the efficacy of the individual components while enabling rapid changes of ingredient composition. Easily and rapidly changing ratios of ingredients can greatly enhance the efficacy and value of the end use composition.

One example is an insect repellent composed of a complex mixture of two or more individual components, with the components being in a form that prevents interaction between the two components, preserving the efficacy of the individual ingredients. This invention also allows rapid changes in the ratio of components resulting in a fast and effective method to create insect repellents that remain effective while reducing the potential for insects to develop resistance to a specific pre-blended composition.

In this invention two or more insect repellents, in a form that prevents or greatly minimizes chemical interaction, are blended together to form a complex mixture. By using components that are in a form that prevents interactions, the composition can be easily changed to create blends that are repellent to various insect species.

One example is to create a blend of microencapsulated essential oils. The essential oils are first microencapsulated individually. The individual microcapsules are then blended together in ratios that achieve repellency to mosquitoes, other insects and arthropods. The repellents can be blended together in the same liquid without forming a mixture with unknown properties.

This concept was developed and tested using a combinations of various essential oil blends and essential oil concentrations. Each test treated textiles fabrics with formulations that included five or six essential oils which had been previously microencapsulated individually and mixed together prior to application. The result is a treated fabric with microcontainers of each individual essential oil which are released using friction.

Essential oils of Citronella, Cedarwood, and Lemongrass are useful as insect repellents. The preferred blend was selected for larger field trials and used on three genera of mosquitos. The test results show that the three blend ratios performed differently, which indicates the need for rapid adjustment of various blend levels that can be achieved easily with this invention.

A plant-based insect repellent according to the present technology may be applied directly to outdoor furniture. When furniture is used, the present technology activates and releases a long-lasting repellent with a pleasant scent. Exemplary systems may be safe for the family, pets, and the environment. Exemplary systems use essential oils as an alternative to DEET-based insect repellents. Synthetic chemicals, such as DEET, can be sticky and unpleasant-smelling when applied to the skin.

An exemplary delivery system enables various blends of repellents (ie. Cedarwood, Citronella and Lemongrass). The interaction between essential oils is reduced by having each individual essential oil encapsulated in a micro-container. Each formulation contains 5-6 different essential oils. Keeping the essential oils separated in micro-containers until activated ensures that the efficacy of each repellent is not changed by chemical interaction with other essential oils and other ingredients in the formulation. Essential oils volatilize when applied directly to a surface, but in the exemplary system they are held in micro-containers or complex matrices that significantly reduce the evaporation rate.

Exemplary embodiments provide a ready to use mixture of plant derived essential oils that have insect repellent properties. The blend of essential oils provides a complex mixture that repels numerous nuisance insects such as mosquitoes, gnats, flies, and ticks. Before the advent of synthetic chemicals, plant extracts and dried plants ground into powders were used to repel insects.

Exemplary embodiments harness the natural power of Lemongrass and other plant essential oils in a consumer-friendly liquid delivery system. Exemplary systems hold the essential oils in micro-containers, reducing evaporation until the product is activated by touch. Stronger fragrances as well as essential oil components are released during use when touch activated. Exemplary systems avoid skin irritation by preventing and/or reducing skin contact with the active ingredient.

An exemplary formulation of an insect repellent may include de-ionized water as a suspension, which may represent 90 to 100 percent of the final product. In further preferred embodiments, the suspension liquid may represent 95 to 100 percent of the final product, and particularly preferably, 97 percent of the final product.

Essential oils of Rosemary, Lemongrass, Citronella, Cedarwood, Geranium, and Thyme may be encapsulated and used in varying concentrations. For example, Rosemary may represent 0.5 percent, Lemongrass may represent 0.6 percent, Citronella may represent 0.5 percent, Cedarwood may represent 0.45 percent, Geranium may represent 0.2 percent, an Thyme may represent 0.05 percent of the final insect repellent product.

Further exemplary embodiments utilize CBD for pain relief. Blends of CBD and other active ingredients can be obtained utilizing the present technology, reducing the interaction of the CBD and other active ingredients until they are released by friction. An exemplary embodiment provides a CBD spray for relieving muscle and/or joint pain and/or discomfort. CBD may be used synergistically with Menthol and Copaiba essential oil. Exemplary embodiments provide a versatile formulation designed with both immediate and encapsulated, delayed-release natural ingredients, to provide immediate relief and longer term pain relief to a user. Exemplary solutions including encapsulated CBD and/or other essential oils may be used treat sleeves and socks that have previously been treated (retreating to recharge the potency), or may also be applied to any close fitting athletic wear. Each active ingredient is separately encapsulated and then blended both for treatment of braces and in a recharge liquid.

Exemplary formulations for textiles and or fabrics include CBD of 250 mg, Copaiba essential oil of 62 mg, and Menthol of 100 mg. Exemplary formulations for sprays for recharging, or charging initially, a sleeve, sock, or other item of athletic wear, include CBD of 1000 mg, Copaiba essential oil of 250 mg, and Menthol of 400 mg. These formulations are merely exemplary and other formulation are possible, in a nearly limitless number of variations, in accordance with the present technology. Mixtures of encapsulated CBD and emulsified CBD (unencapsulated) in a suspension liquid may be used for faster transdermal absorption into the skin, along with a time-release function.

Further exemplary embodiments for pain relief and to improve mobility utilize CBD and Squalene essential oil. The CBD in this situation, as well as in other exemplary embodiments, may be THC Free CBD Isolate. An exemplary dosage may be 250 mg CBD, which may be safely delivered. Each active ingredient may be separately encapsulated and then blended both for treatment of braces or other athletic wear during manufacture, or for sale in a recharge liquid for consumers to treat and re-treat their own athletic wear or personal items. Exemplary formulations for textiles and or fabrics include CBD of 250 mg and Squalene essential oil of 100 mg. Exemplary formulations for sprays for recharging, or charging initially, a sleeve, sock, or other item of athletic wear, include CBD of 1000 mg and Squalene essential oil of 250 mg.

Further exemplary embodiments utilize the present technology in the area of wellness and aromatherapy. Unique blends of multiple essential oils may be used to create relaxing aromatherapy combinations. The present technology utilizes various blends of encapsulated ingredients. Each active ingredient is separately encapsulated and then blended. Exemplary formulations for textiles and or fabrics include Lavender of 750 mg and Benzoin of 1000 mg. Exemplary formulations for sprays for recharging, or charging initially, a personal item include Lavender of 1000 mg and Benzoin of 2000 mg.

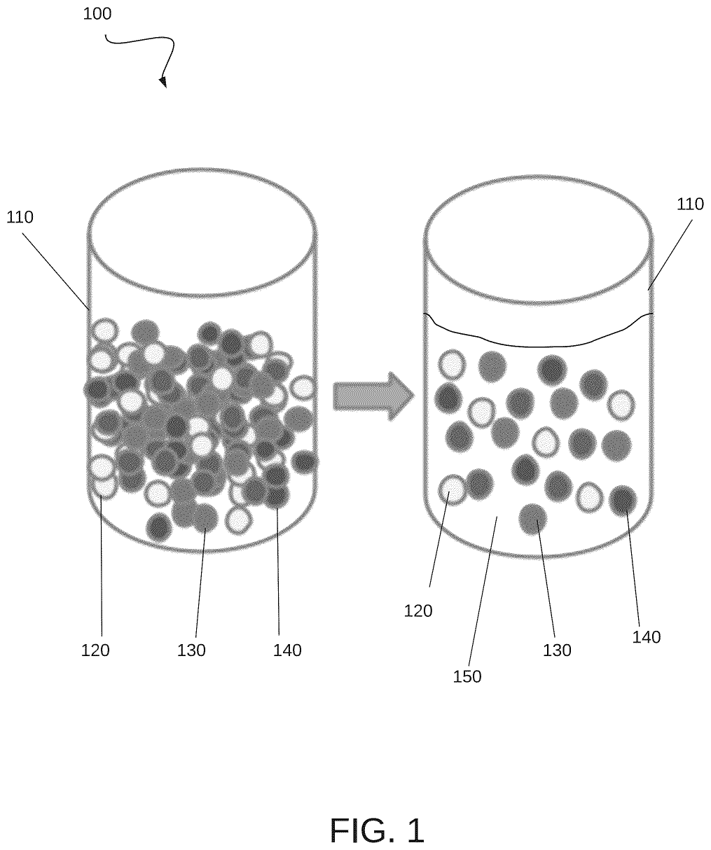

FIG. 1 is diagram 100 illustrating container 110 including chemical encapsulations without a suspension fluid and with a suspension fluid. Container 110 on the left side of FIG. 1 includes three encapsulations, 110, 120, and 130, which represent three distinct active ingredients. The active ingredients in encapsulations 110, 120, and 130 may be in high concentration in container 110. Each of the encapsulations may prevent interaction between the distinct active ingredients contained in encapsulations 110, 120, and 130. The encapsulations may be polymers, and may be designed to remain intact through a period of use, may be designed to rupture when handled directly or sprayed, and/or may be designed to degrade over a period of time to liberate their contents. The right side of FIG. 1 shows container 110 with encapsulations 110, 120, and 130 in a suspension 150. Suspension may be de-ionized water. CBD infused emulsifier, or any other appropriate fluid adapted to hold encapsulations 110, 120, and 130. Suspension 150 may be designed to be inert with respect to encapsulations 110, 120, and 130, or may be designed to slowly degrade encapsulations 110, 120, and 130. Suspension 150 including encapsulations 110, 120, and 130 may be applied directly to skin, may be applied to textiles or other materials during manufacture, and/or may be applied to textiles or other items by a consumer in an after-market scenario. Three encapsulations 110, 120, and 130 are shown in FIG. 1, however, fewer or more encapsulations may be used, for instance, 1, 2, 4, 5, 6 or more separate encapsulations of distinct active ingredients may be used in the present technology.

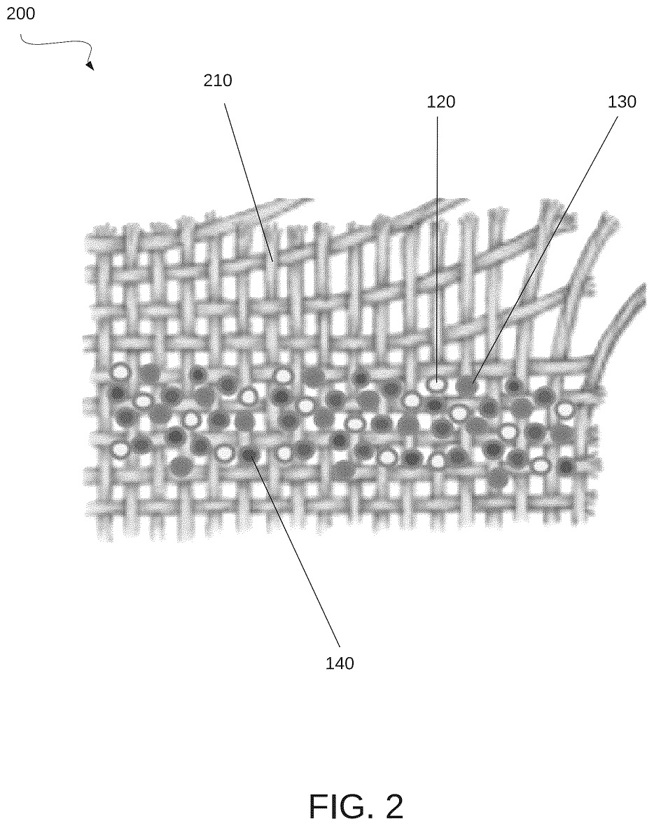

FIG. 2 is a diagram illustrating chemical encapsulations 110, 120, and 130 embedded into fabric 200. Fabric 200 may be any woven fabric, textile, or any other material into which, or onto which, encapsulations 110, 120, and 130 may be applied. Encapsulations 110, 120, and 130 may be embedded into fabric 200 during manufacture or by a consumer, may be applied by spraying, soaking, or any other appropriate method of application. A suspension may be used to assist application and retention of encapsulations 110, 120, and 130 into fabric 200, and the suspension may be washed away, evaporate, or remain on fabric 200.

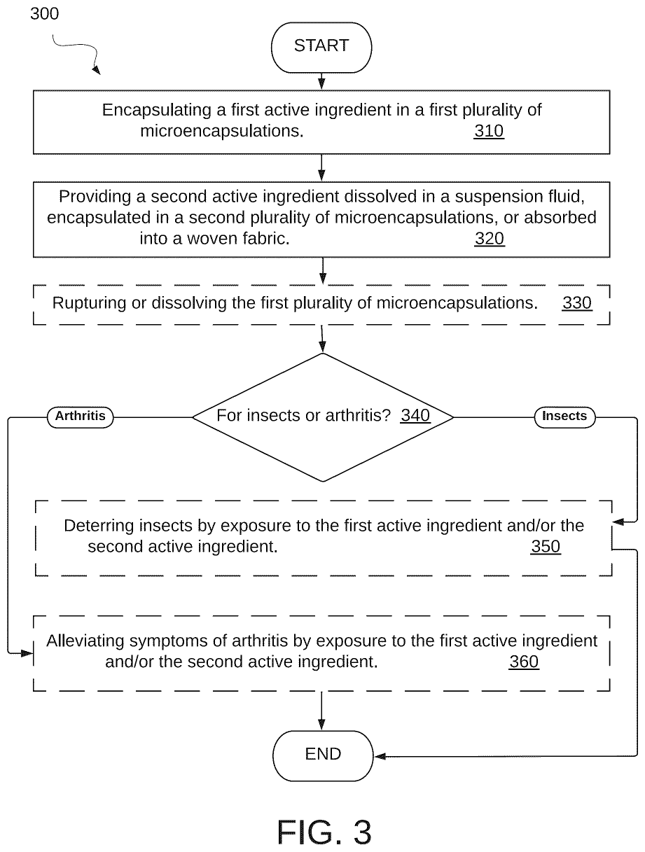

FIG. 3 is a flowchart illustrating method 300 according to an exemplary embodiment of the present invention. Operations shown in dotted lines are optional in method 300. Method 300 begins from the start oval and proceeds to operation 310, which indicates to encapsulate a first active ingredient in a first plurality of microencapsulations. From operation 310, the flow in method 300 continues to operation 320, which indicates to provide a second active ingredient dissolved in a suspension fluid, encapsulated in a second plurality of microencapsulations, or absorbed into a woven fabric. However, any material may be substituted in FIG. 3 for woven fabric and still remain within the scope of the invention. Additional active ingredients may be provided in additional encapsulations. From operation 320, the flow proceeds to optional operation 330, which indicates to rupture or dissolve the first plurality of microencapsulations. From optional operation 330, the flow in method 300 proceeds to decision 340, which asks whether the formulation is an insect repellent or for arthritic pain relief. Numerous alternative uses are disclosed herein and may be substituted for the exemplary usages provided in FIG. 3. If the answer to decision 340 indicates the formulation is for repelling insects, the flow in method 30) proceeds to operation 350, which indicates to deter insects by exposure to the first active ingredient and/or the second active ingredient. From operation 350, the flow proceeds to the end oval. If the answer to decision 340 indicates the formulation is for arthritic pain relief, the flow in method 300 proceeds to operation 360, which indicates to alleviate symptoms of arthritis by exposure to the first active ingredient and/or the second active ingredient. From operation 350, the flow proceeds to the end oval.

The above description is illustrative and not restrictive. Many variations of the technology will become apparent to those of skill in the art upon review of this disclosure. The scope of the technology should, therefore, be determined not with reference to the above description, but instead should be determined with reference to the appended claims along with their full scope of equivalents.

1. A fluid system, comprising:
a first plurality of microencapsulations encapsulating a first active ingredient;
a fluid holding the first plurality of microencapsulations in suspension; and
a second active ingredient in the fluid.

2. The fluid system of claim 1, wherein the first plurality of microencapsulations are configured to one of:
rupture when exposed to friction; and
dissolve upon passage of a period of time.

3. The fluid system of claim 1, wherein the second active ingredient is dissolved in the fluid.

4. The fluid system of claim 1, wherein the second active ingredient is encapsulated in a second plurality of microencapsulations.

5. The fluid system of claim 4, wherein the second plurality of microencapsulations are configured to one of:
rupture when exposed to friction; and
dissolve upon passage of a further period of time.

6. The fluid system of claim 4, further comprising a third active ingredient one of:
dissolved in the fluid; and
encapsulated in a third plurality of microencapsulations.

7. The fluid system of claim 6, wherein the third plurality of microencapsulations are configured to one of:
rupture when exposed to friction; and
dissolve upon passage of another period of time.

8. A system, comprising:
a material;
a first plurality of microencapsulations encapsulating a first active ingredient, the first plurality of microencapsulations one of embedded in the material and applied to a surface of the material; and
a second active ingredient in the material.

9. The system of claim 8, wherein the first plurality of microencapsulations are configured to one of:
rupture when the material is manipulated; and
dissolve upon passage of a period of time.

10. The system of claim 8, wherein the second active ingredient is one of absorbed into the material and applied to the surface of the material.

11. The system of claim 8, wherein the second active ingredient is encapsulated in a second plurality of microencapsulations.

12. The system of claim 11, wherein the second plurality of microencapsulations are configured to one of:
rupture when the material is manipulated; and
dissolve upon passage of a further period of time.

13. The system of claim 11, further comprising a third active ingredient one of:
absorbed into the material; and
encapsulated in a third plurality of microencapsulations.

14. The system of claim 13, wherein the third plurality of microencapsulations are configured to one of:
rupture when the material is manipulated, and
dissolve upon passage of another period of time.

15. The system of claim 8, wherein:
the material is formed into one of a knee brace, a wrist brace, an elbow brace, an ankle brace, a glove, and a sock; and
one of the first active ingredient and the second active ingredient is a THC-free CBD isolate.

16. A method of providing exposure to a first active ingredient and a second active ingredient, comprising:
encapsulating, in a first plurality of microencapsulations, a first active ingredient; and
providing a second active ingredient one of:
dissolved in a fluid holding the first plurality of microencapsulations in suspension:
encapsulated in a second plurality of microencapsulations; and
absorbed into a material in which the first plurality of microencapsulations are embedded.

17. The method of claim 16, wherein the first plurality of microencapsulations are configured to one of:
rupture when applied to a surface; and
dissolve upon passage of a period of time.

18. The method of claim 16, wherein exposure to at least one of the first active ingredient and the second active ingredient operates to alleviate symptoms of arthritis.

19. The method of claim 16, wherein exposure to at least one of the first active ingredient and the second active ingredient operates to repel insects.
